(19) United States
(12) Patent Application Publication
(10) Pub. No.: US 2006/0089723 A1
(43) Pub. Date: Apr. 27, 2006

(54) Method for bone augmentation

(76) Inventor: Kieran P. Murphy, Baltimore, MD (US)

Correspondence Address: TORYS LLP, 79 WELLINGTON ST. WEST, SUITE 3000, TORONTO, ON, M5K 1N2, CA

(21) Appl. No.: 11/254,640

(22) Filed: Oct. 21, 2005

Related U.S. Application Data
(60) Provisional application No. 60/621,060, filed on Oct. 25, 2004.

(51) Int. Cl. A61F 2/28

(52) U.S. Cl. 623/23.62

(57) ABSTRACT

A method for augmenting bone is provided. In an embodiment, a bone cement is administered, typically directly to the affected bone, and a bone augmentation agent administered, via any suitable means including orally, nasally, transdermally. The bone augmentation agent is chosen to complement the bone cement, to thereby facilitate the integration of the bone cement into the natural bone which contacts the bone cement.

### PRIORITY CLAIM

The present application claims priority from U.S. Provisional Patent Application No. 60/621,060 filed Oct. 25, 2004, the contents of which are incorporated herein by reference.

### FIELD OF THE INVENTION

The present invention relates generally to conditions of the bone including osteoporosis and more particularly relates to an apparatus and method for bone augmentation.

### BACKGROUND OF THE INVENTION

Many forms of bone degeneration, fractures, injury and disorders are known. Osteoporosis, a debilitating bone-thinning disease, is an example of such. Osteoporosis currently afflicts over two-hundred million people worldwide. The disease leads to thinner and more fragile bones and an increased susceptibility to fracture. Throughout a lifetime, a person's bone undergoes continuous turnover. Old bone is removed (“bone resorption”) and new bone is added (“bone formation”). Osteoporosis develops as the resorption process overwhelms the formation process, resulting in a net loss in bone density. For example, by age fifty-five, the average woman has already lost thirty percent of her bone mass. Eventually, bone loss can progress to the point where bones become so thin that they are susceptible to fracture from even the slightest trauma.

Osteoporosis is drastically accelerated during menopause and is the third leading cause of death of women over seventy. The disease also afflicts men, who account for twenty percent of all osteoporosis sufferers. By the age of seventy-five, approximately ninety percent of all women and thirty-three percent of all men will suffer from osteoporosis. The ailment causes 1.5 million fractures a year, resulting in annual U.S. health care costs exceeding $14 billion. One in two women and one in eight men over age fifty will have an osteoporosis-related fracture in their lifetime. Of those who suffer from hip fractures, one in five will not survive more than one year. Currently, less than ten percent of afflicted persons are treated for osteoporosis with prescription drugs.

Vertebroplasty and kyphoplasty are procedures for vertebral augmentation that also treat pain associated with vertebral compression fractures. They have become common in the United States of America. Both of these procedures use X-Ray guidance and transpedicular or parapedicular technique to access the vertebral body and inject liquid cement. This cement solidifies to augment the weakened and painful vertebra. The simplest procedure is vertebroplasty and there are seventeen years of experience with this technique. A discussion of vertebroplasty is found in the inventor's U.S. Pat. No. 6,273,916, the contents of which are incorporated herein by reference. A more recent procedure, becoming more common, is kyphoplasty. This involves the inflation of a balloon to restore height. A bone cement is injected into the cavity created by the balloons.

A highly popular bone cement for these procedures is polymethyl methacrylate (“PMMA”). Use of PMMA is described in various papers, including:
(a) “Is Percutaneous Vertebroplasty without Pretreatment Venography Safe? Evaluation of 205 Consecutives Procedures” Cristiana Vasconcelos, Philippe Gailloud, Norman J. Beauchamp, Donald V. Heck, and Kieran J. Murphy, AJNR Am J Neuroradiol 23:913-917, June/July 2002 (“Vasconcelos”);
(b) “Bone Cements: Review of Their Physiochemical and Biochemical Properties in Percutaneous Vertebroplasty” Matthew J. Provenzano, Kieran P. J. Murphy, and Lee H. Riley III AJNR Am J Neuroradiol 25:1286-1290, August 2004 (“Provenzano”);
(c) “The Chemistry of Acrylic Bone Cements and Implications for Clinical Use in Image-Guided Therapy” David A. Nussbaum, M S, Philippe Gailloud, MD, and Kieran Murphy, MD. J Vasc Interv Radiol 2004; 15 Page 1. (“Nussbaum”)
(The contents of these papers are incorporated herein by reference.)

PMMA is an acrylic bone cement. PMMA is not adhesive and it does not integrate into bone over time, and yet it is remarkably strong. As an analogy, PMMA can act like a rebar in cement in building construction. PMMA can, in fact, remove or reduce forces that maintain bone density by supplanting the role of trabecular bone structure in its neighborhood, and thus removing or reducing the electrical charge that contributes bone development.

Unfortunately, the monomer liquid used to dissolve the PMMA powder can be toxic and has been associated with complications such as death and cardiac arrest (See Nussbaum). The high compressive strength of PMMA can cause adjacent vertebral body fractures by exerting high non compliant forces on the adjacent vertebra, as the vertebral body is too stiff as a result of augmentation. These adjacent fractures occur between eight and ten percent of the time.

A promising alternative to PMMA are biologically active bone cements. However, there is currently little knowledge of how to use biologically active bone cement for vertebral augmentation procedures. Biologically active bone cements that integrate into the trabecular bone structure can potentially allow restoration of normal bone in an area of the fracture. This would be advantageous as normal trabecular bone has sophisticated weight distribution, compression shear strength and regenerative properties that cannot be modeled by PMMA.

Biologically active bone cements can also obviate some of the PMMA difficulties. Biologically active bone cements can be of lower strength than PMMA, thus causing less stiffness of the vertebral body when they are injected. However, there are a number of problems with using biologically active bone cement for vertebral augmentation. For example, biologically active bone cements are however very difficult to inject, lack natural radio density, and do not always integrate well for months or even years. Further, some biologically active bone cements require hours before they solidify and become safe. More generally, there have been deaths from the use of some of these cements which may be related to the pH from the cement injected or the leaking of calcium into the circulation resulting in disseminated clotting.

Another type of cement used in vertebral augmentation is calcium phosphate. Calcium phosphate cements are composed of a powder and a liquid solution that dissolves the powder. They are used widely in hip, spine and wrist surgery and also in cranial restriction. There are two different families of calcium phosphates cements. One group undergoes an exothermic reaction and another group undergoes an endothermic reaction. Another group belongs to a family called Bruschite cements. The other group belongs to a family that ultimately form hydroxy appetite, the precursor of bone. When calcium phosphate powders and the aqueous solution are mixed, a paste is formed which sets within minutes to hours. Thus, they are often poorly injectable and poorly visualized under x-ray guidance, making them difficult to use for vertebral augmentation procedures. Further, when they are delivered into the bone, they are acted upon by osteoblasts and osteoclasts in the residual trabecular bone structure. If there is no residual trabecular bone structure the peripheral bone cement may be integrated but central bone cement may remain in its unchanged form, a brittle ceramic of low tensile and compressive strength with potential long term consequences.

### SUMMARY OF THE INVENTION

It is therefore an object of the present invention to provide a novel method for augmenting bone that obviates or mitigates at least one of the disadvantages of the prior art.

The present invention provides a novel apparatus and method to augment bone such as a vertebrae by first injecting a biologically active bone cement into the vertebrae. The bone cement can then be delivered in a radiographically controlled way or by open surgical application. The vertebrae is further augmented by administering a bone augmentation agent. The bone augmentation agent can be administered in any suitable manner, including by injection, intravenously (“IV”), peroral (“PO”), trans-dermally or trans-nasally or transrectally. The bone augmentation agent can be an anabolic bone agent or any other agent that promotes the integration of the injected cement. The bone augmentation agent can help convert the biologically active bone cement to real bone more rapidly then is usually achieved in the poor quality trabecular bone of the osteoporotic patient.

In general, the bone augmentation agent is chosen to complement the bone cement. Thus, some bone cements will be complemented by the use of different combinations of drugs that specifically stimulate its corresponding cement. It is believed that a beneficial effect on cortical bone can also occur in certain circumstances.

The integration of a injected biologically active bone cement into the adjacent trabecular network can be achieved by stimulating the trabecular network and activate osteoblasts. Pulsed or low doses of parathyroid hormone and/or its recombinant analog rhPTH (also known as teripartide) and/or calcitonin can be used to stimulate bone growth, which can stimulate the integration of the bone matrix into trabecular bone, thus speeding up the integration of the bone cement and its creation of a more natural, compliant, realistic and normal bone matrix.

An aspect of the invention includes a method of treating osteoporotic fracture in the body where a biologic material is delivered into the bone and an orally administered drug is used to assist its integration and transformation from its injected state into a material that is akin to normal native bone.

Another aspect of the invention includes a method of treating osteoporotic fracture in the body where a biologic material is delivered into the bone and a nasally administered drug is used to assist its integration and transformation from its injected state into a material that is akin to normal native bone.

Another aspect of the invention includes a method of treating osteoporotic fracture in the body where a biologic material is delivered into the bone and a transdermally administered drug is used to assist its integration and transformation from its injected state into a material that is akin to normal native bone.

Another aspect of the invention includes a method of treating osteoporotic fracture in the body where a biologic material is delivered into the bone and an injected drug is used to assist its integration and trans formation from its injected state into a material that is akin to normal native bone.

Another aspect of the invention includes a method of treating osteoporotic fracture in the body where a biologic material is delivered into the bone and parathyroid hormone (“PTH”) is used to assist its integration and transformation from its injected state into a material-that is akin to normal native bone.

Another aspect of the invention includes a method of treating osteoporotic fracture in the body where a biologic material is delivered into the bone and recombinant parathyroid hormone (“rhPTH”) is used to assist its integration and transformation from its injected state into a material that is akin to normal native bone.

Another aspect of the invention includes a method of treating osteoporotic fracture in the body where a biologic material is delivered into the bone and calcitonin is used to assist its integration and transformation from its injected state into a material that is akin to normal native bone.

Another aspect of the invention includes a method of treating osteoporotic fracture in the body where a biologic material is delivered into the bone and growth hormone is used to assist its integration and transformation from its injected state into a material that is akin to normal native bone.

Another aspect of the invention includes a method of treating osteoporotic fracture in the body where a biologic material is delivered into the bone and insulin related growth factor is used to assist its integration and transformation from its injected state into a material that is akin to normal native bone.

Another aspect of the invention includes a method of treating osteoporotic fracture in the body where a bone cement composed of Calcium phosphate is delivered into the bone and a integration stimulant used to assist its integration and transformation from its injected state into a material that is akin to normal native bone.

Another aspect of the invention includes a method of treating osteoporotic fracture in the body where a bone cement composed of Calcium hydroxyapetite is delivered into the bone and an integration stimulant used to assist its integration and transformation from its injected state into a material that is akin to normal native bone.

Another aspect of the invention includes a method of treating osteoporotic fracture in the body where a bone cement composed of Calcium sulphate is delivered into the bone and an integration stimulant used to assist its integration and transformation from its injected state into a material that is akin to normal native bone.

Another aspect of the invention includes a method of treating osteoporotic fracture in the body where a bone cement composed of Calcium aluminate is delivered into the bone and an integration stimulant used to assist its integration and trans formation from its injected state into a material that is akin to normal native bone.

Another aspect of the invention includes a method of treating osteoporotic fracture in the body where a bone cement composed of bone morphogenic protein is delivered into the bone and a integration stimulant used to assist its integration and trans formation from its injected state into a material that is akin to normal native bone.

Another aspect of the invention includes a method of treating vertebral fracture using cement that has an insulin related growth factor (“IGF”) embedded therein, and which is delivered into the vertebrae. The method also includes administering calcitonin or PTH or other bone augmentation accelerant using any suitable delivery mechanism, such as orally, nasally, injection, transdermally etc.

Another aspect of the invention includes a method of treating osteoporotic fracture in the body where a biologic material is delivered into the bone and recombinant parathyroid hormone (“rhPTH”) is also delivered into the bone with the cement as an accelerant of local bone growth and cement integration and transformation from its injected state into a material that is akin to normal native bone.

Another aspect of the invention includes a method of treating osteoporotic fracture in the body where a biologic material is delivered into the bone and recombinant parathyroid hormone (“rhPTH”) and Insulin related growth factor are also delivered into the bone with the cement as an accelerant of local bone growth and cement integration and transformation from its injected state into a material that is akin to normal native bone.

Another aspect of the invention includes a method of treating osteoporotic fracture in the body where a biologic material is delivered into the bone and recombinant parathyroid hormone (“rhPTH”) and Insulin related growth factor are also delivered into the bone with the cement as an accelerant of local bone growth and cement integration and transformation from its injected state into a material that is akin to normal native bone. At the same time Oral/IV or some other method of systemic bone anabolic stimulant is delivered to the patient for a broader increase in global bone density.

### BRIEF DESCRIPTION OF THE DRAWINGS

The present invention will now be explained, by way of example only, with reference to certain embodiments and the attached Figures in which:

FIG. 1 is a flowchart depicting a method for augmenting bone in accordance with an embodiment of the invention;

FIG. 2 shows a representation of an axial view of a vertebra with an osteoporotic fracture, wherein the vertebra is undergoing administration of a bone cement in the performance of one of the steps of FIG. 1;

FIG. 3 shows a representation of the administration of a bone augmentation agent in the performance of one of the steps of FIG. 1; and,

FIG. 4 shows a representation of an axial view of a vertebra with an osteoporotic fracture, wherein the vertebra is undergoing administration of a bone cement in the performance of the steps of FIG. 1 in accordance with an alternate embodiment of the invention.

### DETAILED DESCRIPTION OF THE INVENTION

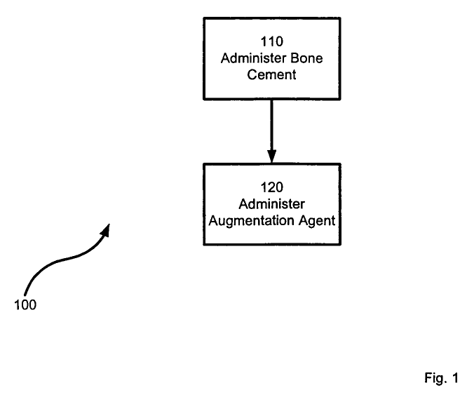

Referring now to FIG. 1, a flowchart depicting a method of augmenting bone is indicated generally 100. Method 100 can be applied to many different types of bone conditions, including degeneration, fractures, injury and disorders. In a presently preferred embodiment, method 100 is used to treat an osteoporotic fracture.

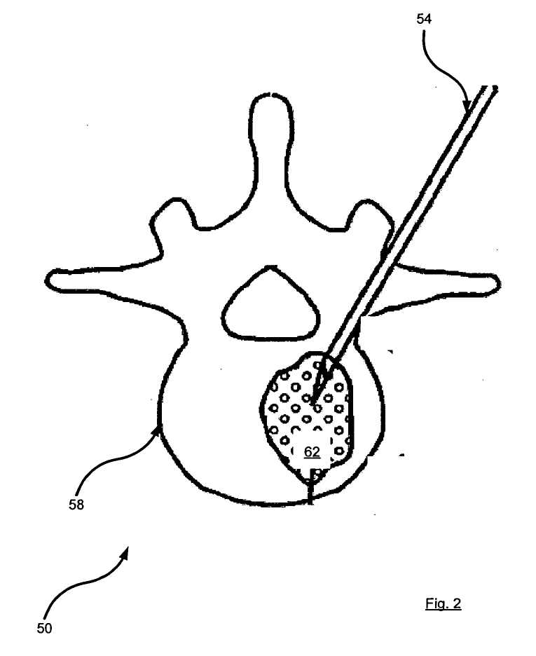

Referring to FIG. 2, a vertebra is indicated generally at 50. Vertebra 50 has an osteoporotic fracture and exemplifies one type of bone (a vertebra) and a corresponding condition (fracture) that can be treated using method 100. Thus, to treat vertebra 50 using method 100, first step 110 would be performed. At step 110, a bone cement is administered. In the case of vertebra 50, such an administration of bone cement is performed using vertebroplasty or kyphoplasty. In FIG. 2, vertebra 50 is shown undergoing a vertebroplasty, in order to effect step 110. Thus, FIG. 2 shows a vertebroplasty needle 54 inserted along a transpedicular approach with the tip of needle 54 positioned within the vertebral body 58. Needle 54 is also shown expressing a bone cement 62 within vertebral body 58.

It should thus be understood that the performance of step 110 on vertebra 50 can be effected using any presently known or future contemplated vertebroplasty techniques, using appropriate or desired needles, image guidance modalities, and the like. The type of bone cement 62 will influence such choices, and will also be chosen to complement the choices used to effect step 120.

In a present embodiment it is contemplated that bone cement 62 is a biologic bone cement, also referred to as a biologically active bone cement. Suitable biologic bone cement is at least one of calcium phosphate, calcium hydroxyapetite, calcium sulphate, calcium aluminate or a bone morphogenic protein, or a combination thereof. It can be desired to select combinations of such cements where one or more cements provides short term stability and/or pain relief, while another provides long term integration and new bone development. A presently preferred biologic bone cement is Hydroxy appetite such as Cementek and Cementek LV (manufactured by Teknimed SA, Toulouse France). Next, at step 120, an augmentation agent is administered. The particular augmentation agent, and the means by which it is administered will typically complement the type of bone cement 62, and the means by which it was administered, at step 110. The augmentation agent is chosen to stimulate integration and transformation of bone cement 62 into a material that is at least substantially similar to the normal native bone that would otherwise be present within vertebral body 58 in its normally healthy state.

In one embodiment the bone augmentation agent is the bone anabolic agent known as truncated PTH[1-34] in the free acid form. PTH[1-34] is commercially available in an FDA-approved pharmaceutical formulation from Eli Lilly & Co. under the brand name Forteo®. Other bone anabolic agents include, but are not limited to, an amidated truncate of natural parathyroid hormone, PTH[1-30]NH₂, PTH[1-31]NH₂, PTH[1-32]NH₂, PTH[1-33]NH₂, PTH[1-34]NH₂ and combinations thereof In a preferred embodiment the bone anabolic agent is PTH[1-34]NH₂.

Methods for the preparation of truncated parathyroid hormones are described in U.S. Pat. No. 6,103,495 to Mehta et al. Methodologies for amidating such truncated parathyroid hormones are provided in, for example, U.S. Pat. No. 5,789,234 to Bertelson et al., and U.S. Pat. No. 6,319,685 to Gilligan et al.

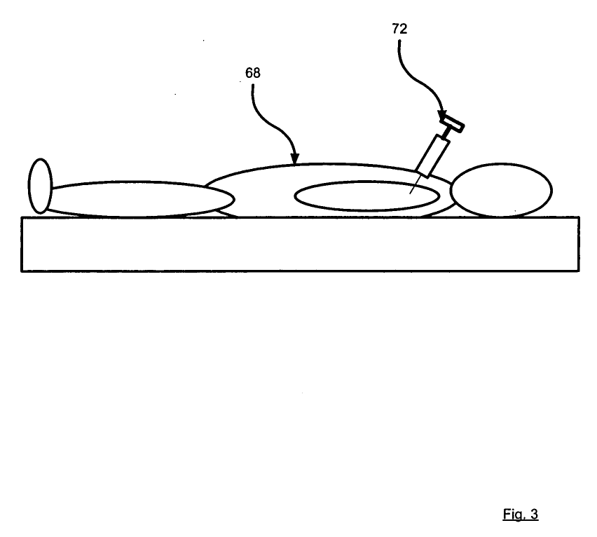

Step 120 is represented in FIG. 3, as the patient 68 suffering from the fracture of vertebra 50 is depicted in recovery having a bone augmentation agent in the form of the bone anabolic agent PTH[1-34]NH₂ being administered via an injection through needle 72.

In an embodiment, a sufficient amount of PTH[1-34]NH₂ is administered to patient 68 via needle 72 to achieve, and maintain, a pulsatile blood concentration thereof in patient 68 of between about 50 and about 350 pg/ml, preferably between about 100 and about 200 pg/ml, and most preferably between about 150 pg/ml. In another embodiment, the blood concentration of the PTH[1-34]NH₂ in patient 68 is raised to its preferred level by no later than seven days following the performance of step 110. As is well understood by those skilled in the art, an appropriate dosage of PTH[1-34]NH₂ is determined to achieve the desired blood concentrations. In the case of injection formulations thereof via needle 72, can, though need not necessarily be, in the range of between about 10 to about 200 micrograms (“μg”), given once per day, more preferably between about 20 and about 100 μg per does and more preferably between about 20 and about 50 μg per dose, or most preferably between about 20 and about 40 micrograms per dose given once per day. Dosage levels of injectable formulations comprising bone augmentation agents other than PTH[1-34]NH₂ would be consistent with those noted above.

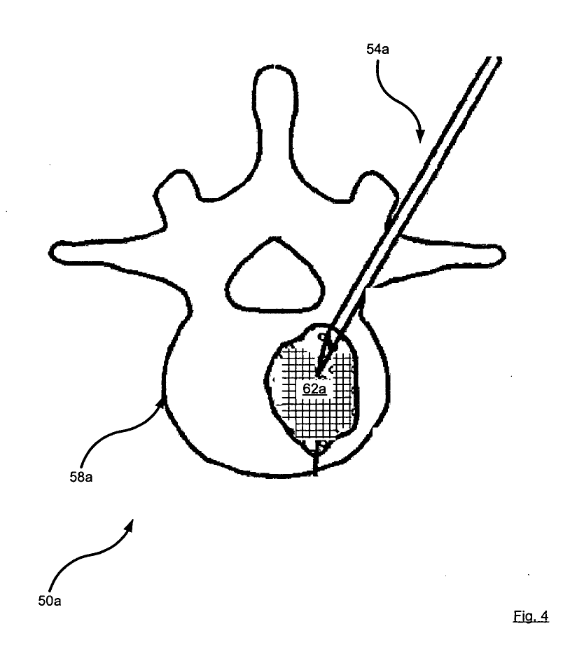

Other means of implementing method 100 are contemplated. Referring now to FIG. 4, a vertebra is indicated generally at 50a. Vertebra 50a also has an osteoporotic fracture and exemplifies one type of bone (a vertebra) and a corresponding condition (fracture) that can be treated using method 100. However, in this embodiment, to treat vertebra 50a using method 100, step 110 and step 120 are performed substantially simultaneously. FIG. 4 shows an enhanced bone cement 62a. Enhanced bone cement 62a includes a bone cement, as well as an augmentation agent. Examples of the bone cement used in enhanced bone cement 62a include Cementek and Cementek LV (manufactured by Teknimed SA, Toulouse France) while examples of the augmentation agent used in enhanced bone cement 62a include insulin related growth factor (“IGF”), rhPTH, GH, anabolic vitamin D analogs, low density lipoprotein receptor related protein 5 (LRP5), an activator of non genomic estrogen signaling (ANGELS), a bone morphogenic protein, a growth hormone releasing factor (GHRF) hepatcyte growth factor (HGF) calcitonin gene related peptide (CGRP) parathyroid related peptide (PTHrP) Transforming growth factor (TGF)_and/or combinations thereof. Thus, FIG. 2 shows a vertebroplasty needle 54a inserted along a transpedicular approach with the tip of needle 54a positioned within the vertebral body 58a. Needle 54a is also shown expressing the enhanced bone cement 62a within vertebral body 58a.

As still another means of implementing method 100, the administration of the bone cement and the augmentation agent can be done via separate injections into the vertebral body. Thus the cement may be injected via one pedicle of the vertebral body and the accelerant of bone integration could be injected by the other pedicle or through an ipsilateral approach via the same pedicle as the cement, or mixed with the cement.

While only specific combinations of the various features and components of the present invention have been discussed herein, it will be apparent to those of skill in the art that desired subsets of the disclosed features and components and/or alternative combinations of these features and components can be utilized, as desired.

Likewise, other bone augmentation agents can be used at step 120 such as calcitonin, growth hormone releasing factors, Insulin or glucagon. Such an agent will typically be chosen to complement the cement used at step 110. Further, while the embodiments herein discuss the administration of the bone augmentation agent via injection, it is contemplated that the bone augmentation agent can be administered in any desired delivery mechanism, such as orally, nasally, transdermally, anally, in accordance with techniques that suitably correspond with the particular agent.

Likewise, it should also be understood that the steps in method 100 can be performed in a different order than shown, or simultaneously. Where performed separately, the time between performance of each step in method 100 can vary, as suitable, according to the chosen bone cement and augmentation agent.

Likewise, other bones can be augmented using the teachings herein, including, but not limited to, the proximal femoral, the distal radius, the proximal humerus, the calcaneus, a rib, or ribs, the tibia, or the sacrum.

The above-described embodiments of the invention are intended to be examples of the present invention and alterations and modifications may be effected thereto, by those of skill in the art, without departing from the scope of the invention which is defined solely by the claims appended hereto.

The foregoing specification makes certain references to various third party documents including publications, patents, and the like. The contents of all such documents are incorporated herein by reference.

1. A method for augmenting bone comprising the steps of:
administering a bone cement;
administering a bone augmentation agent complementary to said bone cement.

2. The method of claim 1 wherein said bone cement is a biologic material delivered into the bone and said bone augmentation agent is parathyroid hormone (“PTH”).

3. The method of claim 1 wherein said bone cement is selected from the group consisting of a biologic material, including calcium phosphate, calcium hydroxyapetite, calcium sulphate, calcium aluminate, a bone morphogenic protein.

4. The method of claim 1 wherein said bone augmentation agent is selected from the group consisting of calcitonin, growth hormone, insulin related growth factor, PTH.

5. The method of claim 1 wherein said bone augmentation agent is the pharmaceutical formulation from Eli Lilly & Co. sold under the brand name Forteo®.

6. The method of claim 1 wherein said bone augmentation agent is an amidated truncate of natural parathyroid hormone, selected from the group consisting of PTH[1-30]NH₂, PTH[1-31]NH₂, PTH[1-32]NH₂, PTH[1-33]NH₂, PTH[1-34]NH₂ or combinations thereof.

7. The method of claim 1 wherein said bone is a vertebral body.

8. The method of claim 7 wherein said bone cement is selected from the group consisting of calcium phosphate, calcium hydroxyapetite, calcium sulphate, calcium aluminate, a bone morphogenic protein.

9. The method of claim 8 wherein said bone augmentation agent is a sufficient amount of PTH[1-34]NH₂ administered via injection to achieve a pulsatile blood concentration thereof of between about 50 and about 350 pg/ml.

10. The method of claim 8 wherein said bone augmentation agent is a sufficient amount of PTH[1-34]NH₂ administered via injection to achieve a pulsatile blood concentration thereof between about 100 and about 200 pg/ml.

11. The method of claim 8 wherein said bone augmentation agent is a sufficient amount of PTH[1-34]NH₂ administered via intravenous to achieve a pulsatile blood concentration thereof of about 150 pg/ml.

12. The method of claim 8 wherein a blood concentration of PTH[1-34]NH₂ is raised to a desired level by no later than seven days following the performance of said step of administering said bone cement.

13. The method of claim 8 wherein said bone augmentation agent is PTH[1-34]NH₂ which is administered via injection in the range of between about 10 to about 200 micrograms (“μg”), given once per day.

14. The method of claim 8 wherein said bone augmentation agent is PTH[1-34]NH₂ which is administered via injection in the range of between about 20 and about 100 micrograms per dose given once per day.

15. The method of claim 8 wherein said bone augmentation agent is PTH[1-34]NH₂ which is administered via injection in the range of between about 20 and about 50 micrograms per dose given once per day.

16. The method of claim 8 wherein said bone augmentation agent is PTH[1-34]NH₂ which is administered via injection in the range of between about 20 and about 40 micrograms per dose given once per day.

17. The method of claim 8 wherein said bone cement is administered via a vertebroplasty.

18. The method of claim 1 wherein said bone cement is administered via a injection into the proximal femoral.

19. The method of claim 1 wherein said bone cement is administered via a injection into the distal radius

20. The method of claim 1 wherein said bone cement is administered via a injection into the proximal humerus.

21. The method of claim 1 wherein said bone cement is administered via a injection into the calcaneus.

22. The method of claim 1 wherein said bone cement is administered via a injection into the ribs.

23. The method of claim 1 wherein said bone cement is administered via a injection into the tibia.

24. The method of claim 1 wherein said bone cement is administered via a injection into the sacrum.

25. The method of claim 1 wherein said bone augmentation agent is administered orally, nasally, or trans-dermally, rectally, subcutaneously or intravenously.

26. The method of claim 1 wherein said bone cement and said bone augmentation agent are administered substantially simultaneously.

27. The method of claim 1 wherein said bone cement and said bone augmentation agent are administered substantially simultaneously into the vertebral body or other bone being augmented.

28. The method of claim 1 wherein said bone cement and said bone augmentation agent are administered substantially simultaneously into the bone and simultaneous systemic stimulant is also delivered.

29. The method of claim 1 wherein said bone augmentation agent is administered prior to said bone cement.

30. The method of claim 1 wherein said bone augmentation agent is administered in conjunction with said bone cement.

31. A method of treating osteoporotic fracture in a vertebrae comprising the steps of delivering a bone cement and delivering a biologic material into the vertebrae and delivering a recombinant parathyroid hormone (“rhPTH”).

32. A method of treating osteoporotic fracture in a vertebrae comprising the steps of delivering a biologic material into the vertebrae and delivering a recombinant parathyroid hormone (“rhPTH”) and Insulin related growth factor into the vertebrae.

33. A method of treating osteoporotic fracture in a vertebrae comprising the steps of: delivering a biologic material into the bone and a recombinant parathyroid hormone (“rhPTH”) and Insulin related growth factor and administering a systemic bone anabolic stimulant.
